(19) United States
(12) Patent Application Publication
(10) Pub. No.: US 2019/0105191 A1
(43) Pub. Date: Apr. 11, 2019

(54) FIN SLEEVES FOR A MANDIBULAR ADVANCEMENT DEVICE AND METHODS OF USING THE SAME

(71) Applicant: PROSOMNUS SLEEP TECHNOLOGIES, INC., Pleasanton, MA (US)

(72) Inventors: Kim SUNG, Pleasanton, CA (US); David W. KUHNS, Pleasanton, CA (US); Leonard A. LIPTAK, Pleasanton, CA (US)

(73) Assignee: PROSOMNUS SLEEP TECHNOLOGIES, INC., Pleasanton, CA (US)

(21) Appl. No.: 16/088,325

(22) PCT Filed: Jan. 29, 2017
(86) PCT No.: PCT/US17/15530

§ 371 Date: Sep. 25, 2018

Related U.S. Application Data
(60) Provisional application No. 62/289,131, filed on Jan. 29, 2016.

(51) Int. Cl. A61F 5/56; A61C 5/00; A61C 7/00; A61C 7/36

(52) U.S. Cl. CPC A61F 5/566; A61C 5/007; A61F 2005/563; A61C 7/36; A61C 7/002

(57) ABSTRACT

Disclosed herein are sleeves for use with a fin of a mandibular advancement device (MAD), the sleeve comprising: a shell, having a wall defining a hollow interior, wherein the wall encloses the hollow interior on all sides except one, leaving an opening at one end of the body; the wall has a thickness in the range of from about 1 nm to about 5 mm; the hollow interior comprises approximately the same size and dimensions as the fin of the MAD. Also disclosed are MADs that use the sleeves. Also disclosed are methods of treating a patient using MADs that use the sleeves.

### CROSS-REFERENCE TO RELATED APPLICATIONS

This application claims the benefit of U.S. Provisional Application No. 62/289,131, filed 29 Jan. 2016, the entire disclosure of which is incorporated by reference herein.

### FIELD OF THE INVENTION

The present invention is in the field of medical devices, and in particular it is in the field of mandibular advancement devices

### BACKGROUND OF THE DISCLOSURE

Snoring and mild sleep apnea are generally thought to be the result of a reduced or partial constriction of the airway during sleep. This may be attributed to soft tissue sinking and applying pressure on the airway during sleep including the mandible dropping and moving backwards. Mandibular advancement devices are designed to move the mandible forward to relieve the force applied from soft tissue during sleep and assisting in opening the airway. Depending on the patient, the advancement of the mandible may vary based on the response of the patient. Optimizing the adjustment is referred to as titration.

Many devices are on the market today that serve to advance the mandible. A dual arch device comprising fins and an adjustment screw block are represented by Somnomed's Somnodent (U.S. Pat. No. 6,604,527), the Dynflex Dorsal Appliance, and Dr. Nordstrom's NorSnor II produced by Murdock Labs since the 1980's. Each device has an adjustable upper screw mechanism with block, which upon adjustment applies a force against a lower fin or bite block that then moves the mandible forward. The threaded screw system, requires that the user apply an Allen wrench to adjust the screw for titration. These devices are handmade and built up using polymethylmethacrylate (PMMA) and the corresponding monomer, methylmethacrylate. The device is cured in the dental lab and custom designed for each patient. Reproduction of the quality and accuracy of the device is subject to human skill.

Alternatively, there are devices that make an adjustment by changing out straps of different lengths to titrate the mandible forward. Examples of these are the Silent Nite (U.S. Pat. No. 5,365,945) produced by Glidewell, and the Narval (U.S. Pat. No. 7,146,982) produced by Resmed. These devices offer a possibly lower profile than the screw adjustment devices and also connect the upper and lower arch together.

Several limitations exist with these existing sleep apnea devices. First, the manual, artisanal fabrication method used by these devices limits the ability to consistently and precisely transfer the prescription into the device design. This limitation results in the need for additional adjustments. Second, the manual artisanal fabrication method requires the layering of monomers, which can subject the patient to higher levels of residual monomers. Third, existing device designs feature multiple components, straps, and screws that decrease durability, reliability, and ease of use. Fourth, the materials used in the manual, artisanal fabrication method require the device to be of a certain size to effectively withstand the normal intraoral forces that the device is subjected to. This size limitation results in decreased patient comfort and patient compliance.

Therefore, a need exists to provide a mandibular advancement device that provides advancement of the lower jaw through a series of splints digitally designed and milled to provide accurate increments of advancement for easy titration of the mandible, without the need for an adjustment screw mechanism or adjustment straps.

### SUMMARY OF THE INVENTION

Disclosed herein are sleeves for use with a fin of a mandibular advancement device (MAD), the sleeve comprising: a shell, having a wall defining a hollow interior, wherein the wall encloses the hollow interior on all sides except one, leaving an opening at one end of the body; the wall has a thickness in the range of from about 1 nm to about 5 mm; the hollow interior comprises approximately the same size and dimensions as the fin of the MAD. Also disclosed are MADs that use the sleeves. Also disclosed are methods of treating a patient using MADs that use the sleeves.

### BRIEF DESCRIPTION OF THE DRAWINGS

FIG. 1 shows an embodiment of a splint having sleeveless fins.

FIG. 2 shows an embodiment of a splint, having a sleeved fin and a sleeveless fin.

FIG. 3A shows an end view of an embodiment of a sleeve. FIG. 3B shows side, upright, view of an embodiment of a sleeve. FIG. 3C shows a side view of an embodiment of a sleeve. FIG. 3D shows another side view of an embodiment of a sleeve.

FIG. 4A illustrates an embodiment of the sleeve design in a library of sleeves. FIG. 4B illustrates another embodiment of the fin design in a library of sleeves. FIG. 4C illustrates another embodiment of the fin design in a library of sleeves. FIG. 4D illustrates another embodiment of the fin design in a library of sleeves. FIG. 4E illustrates that a sleeve design from the library is incorporated into an embodiment of the disclosed mandibular advancement device.

FIG. 5 shows the bottom view of an embodiment of the sleeve.

FIG. 6A shows the placement of an embodiment of the key-tab locking mechanism on the sleeve. FIG. 6B shows an embodiment of a key tab locking mechanism when the sleeve is placed over the fin. FIG. 6C shows an embodiment of releasing the locking mechanism.

FIG. 7A shows an embodiment of the fin having a key button. FIG. 7B shows an embodiment of the fin to be used with the key button mechanism.

### DETAILED DESCRIPTION OF THE EMBODIMENTS

Mandibular advancement devices have been disclosed previously in the U.S. application Ser. No. 14/809,208 (“the '208 application”), and the corresponding International Application No. PCT/US2014/072398, the entire disclosure of both of which is hereby incorporated by reference in their entirety, including all the drawings. The definitions of many of the terms used herein can be found in the '208 application.

In one aspect provided herein is a digitally designed and milled mandibular advancement device comprising an upper splint and a lower splint, wherein the upper and lower splints independently further comprise one or more fins. Also disclosed, to be used with the device, are a plurality of sleeves, each pair of sleeves having a unique thickness and/or rake angle, and where the sleeves fit over the fins.

Thus, disclosed herein are sleeves for use with a fin of a mandibular advancement device, the sleeve comprising:
a shell, having a wall defining a hollow interior,
   wherein the wall encloses the hollow interior on all sides except one, leaving an opening at one end of the body;
the wall has a thickness in the range of from about 1 nm to about 5 mm;
the hollow interior comprises approximately the same size and dimensions as the fin of the mandibular advancement device.

In some embodiments, the sleeved fins on the splints provide accurate increments of advancement of the lower jaw for titration of the mandible. The terms “dental splint” and “splint” as used herein refers to several types of orthodontic devices that are designed to address dental problems such as loose teeth and bruxism, in addition to problems with snoring and apnea. More specifically, the term “splint” refers to an upper or lower splint, having sleeveless fins, which splint is uniquely designed to fit over a patient's dentition. Thus, as is disclosed further below, the present disclosure distinguishes between a “sleeveless-fin splint,” which is a splint that fits over the patient's dentition but the upper and lower fins do not make sufficient contact to provide the desirable extent of mandibular advancement, and “sleeved-fin splint,” where presently disclosed sleeves have been placed over the upper and lower fins of the splints, where the increased thickness afforded by the sleeves causes mandibular advancement when the splints are worn by the patient.

A patient in need of the disclosed mandibular advancement devices wears the upper splint on the upper dentition and the lower splint on the lower dentition during sleep. The splints are designed to remain attached to the dentition until the patient removes them. The sleeved fins of the upper and lower splints cause a precise placement of the mandible in relation to the maxilla. The mandible is caused to stay in a forward position and does not relax and fall back. The airway constriction during the sleep is thereby minimized.

In one embodiment the device creates an offset between the upper and lower splints by using upper and lower sleeved fins as boundary surfaces to restrict movement while the mouth is closed or reasonably opened, e.g., opened to the same extent that the mouth opens during sleep.

In some embodiments, the splints with sleeveless fins, disclosed herein were digitally designed and then milled as a single unit. In some of these embodiments, a computer aided design (CAD) process were used to design and manufacture the mandibular advancement devices disclosed herein. Plaster models of the patient's upper and lower dental impressions were first obtained using well-known techniques in the art. Then, scans of the plaster models were imported into the CAD software. In other embodiments, the 3D files of the patient's impression are imported from other sources, such as a direct scan of the patient dentition using an Intra Oral Scan (IOS) Device, e.g., the 3M TruDef™ scanner, or a direct scan of the impression from either an IOS or Cone Beam Computed Tomography (CBCT) device. In these embodiments, the files enable the design of the mandibular advancement splint in 3D space in a CAD software such as 3-Matic by Materialise™.

In other embodiments, the different components of the disclosed devices, for example the splint, the fins, the fin sleeves, the retention arms, etc., are milled or manufactured separately and then attached together after the manufacturing. This approach allows for the use of interchangeable parts. The design and manufacturing processes are described in the co-pending U.S. application Ser. No. 15/416,715, the entire disclosure of which, including the drawings, and especially Paragraphs [0012]-[0053], inclusive, are hereby incorporated by reference.

In some embodiments, a unique single set of upper and lower splints with sleeveless fins are prepared for each patient. The patient is then provided with a library of sleeves that fit over the fins. By changing the sleeves, the patient or the healthcare provider can change the extent of mandibular advancement. This approach to the manufacture and use of mandibular advancement devices provides for a less costly, easier to use, and easier to manufacture approach to mandibular advancement.

In one embodiment, the splint is designed to comfortably fit on to a patient's upper and lower arches, and maintain a maximum amount of space for the tongue. The sleeved fin keeps the mandible advanced forward per a doctor's prescription while still allowing the patient to reasonably open their mouth and move their jaw from left to right for comfort. This contact serves as a barrier to keep the lower sleeved fin in a position forward of this fin engagement surface.

The disclosed devices can be made from any material that can withstand the oral environment for an extended period of time, for example overnight. Furthermore, the material can be any material that is capable of being milled to form the devices disclosed herein. Examples of materials include plastics and other polymers, whether hard or soft, transparent or opaque. Some suitable polymers include, but are not limited to, a polyetheretherketone (PEEK), polystyrene, polyvinyl chloride, rubber, synthetic rubber, or an acrylate polymer, such as a polymer made up of methyl methacrylate, methyl acrylate, ethyl acrylate, 2-chloroethyl vinyl ether, 2-ethylhexyl acrylate, hydroxyethyl methacrylate, butyl acrylate, butyl methacrylate, or trimethylolpropane triacrylate (TMPTA).

Embodiments of the device are further described with reference to the accompanying drawings. While the below discussion is in the context of a sleeve for a lower splint 100, it is explicitly contemplated that the same aspects and embodiments are also applicable to an upper splint.

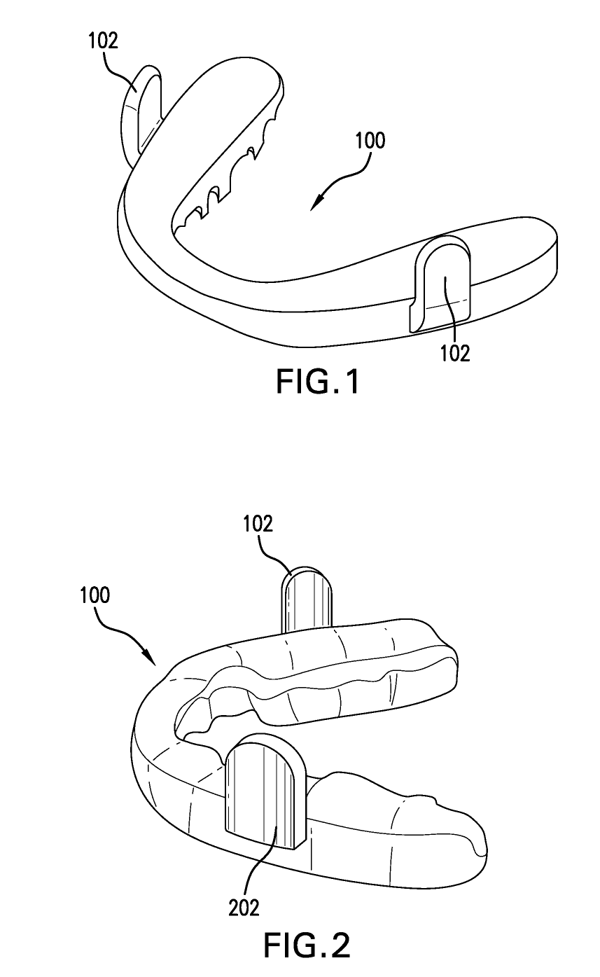

Referring now to FIG. 1, a lower splint 100 is shown with sleeveless fins 102. In some embodiments, both the upper and lower splints comprise sleeveless fins configured to receive a sleeve. In other embodiments, one of the upper or lower splint comprises sleeveless fins configured to receive a sleeve, while the other of the upper or lower splint is configured to make contact with the sleeved fin of the other splint. That is, the fin thickness and/or rake angle of one splint can be varied while the fin thickness and/or rake angle of the other splint is kept constant. While the disclosure here is in the context of the fins on a lower splint, the skilled artisan recognizes that both the upper and the lower splints, or either of the upper or lower splints, can be made to exhibit the use of the sleeves disclosed herein.

FIG. 2 shows an embodiment of the lower splint 100. The right fin 102 is a sleeveless fin that is configured to receive a fin. The left fin 202 is a sleeved fin, where a sleeve has been placed over the fin. The sleeve increases the thickness of the fin and can provide a rake angle that is different than that of the fin, or that of other sleeves. In the embodiment shown in FIG. 2, the rake angle of both the fin 102 and the sleeved fin 202 is neutral. In some embodiments, the rake angle is 90°, while in other embodiments, the rake angle is between about 20° to about 80°, for example, an angle selected from the group consisting of about 20°, about 25°, about 30°, about 35°, about 40°, about 45°, about 50°, about 55°, about 60°, about 65°, about 70°, about 75°, and about 80°. In other embodiments, the rake angle is between about 100° to about 160°, for example, an angle selected from the group consisting of about 100°, about 105°, about 110°, about 115°, about 120°, about 125°, about 130°, about 135°, about 140°, about 145°, about 150°, about 155°, and about 160°.

By “about” a certain value it is meant that the stated value comprises the range of values within ±25%, ±20%, ±10%, or ±5% of the stated value. Thus, by way of example only, if a distance is given as “about 5 mm,” the range of distances between 3.75 mm (5−25%) to 6.25 mm (5+25%) is envisioned.

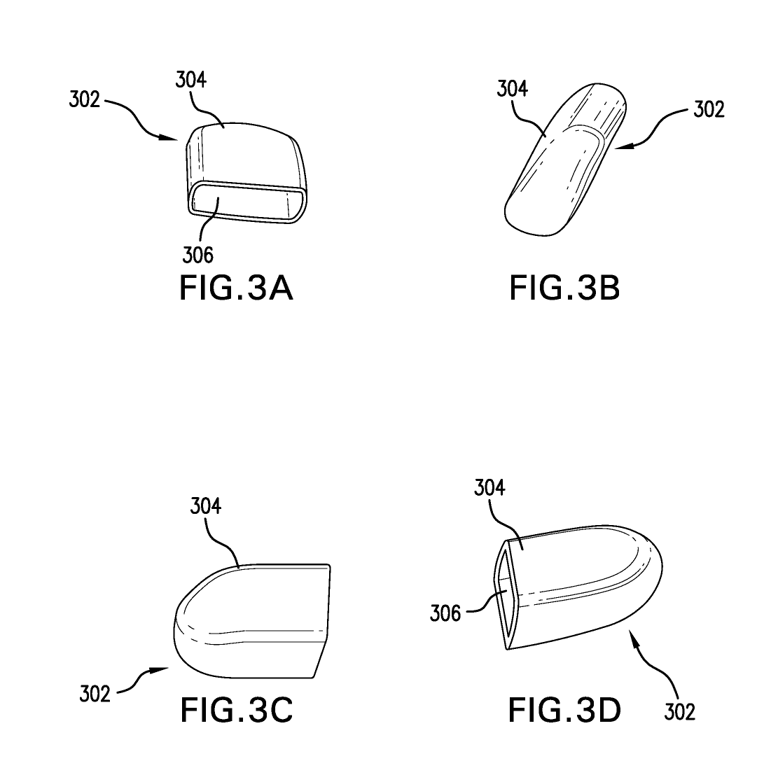

FIGS. 3A-3D show various views of an embodiment of a sleeve 302. Each sleeve 302 comprises an outer shell 304, which defines a hollow interior 306. Thus, the sleeve 302 has an open end, where the hollow interior is accessed, and a closed end opposite the open end. The dimensions of the hollow interior 306 are such that the sleeveless fin 102 fits inside the hollow interior 306. When worn over the fin 102, the open end of the sleeve 302 abuts the splint 100, whereas the closed end is distal to the splint 100.

In some embodiments, the fit between the sleeve 302 and the fin 102 is such that when the sleeve 302 is placed over the fin 102, the sleeve 302 is substantially immobile with respect to the fin 102. By “substantially immobile” it is meant that the movement of the sleeve 302 with respect to the fin 102 is not perceptible by the naked eye (that is to say, the sleeve 302 does not “rattle” when it is placed over the fin 102).

In some embodiments, the closed end of the sleeve 302 is curved away from the plane normal to the plane defined by the rim of the open end. This angle is shown in FIG. 3B, where the closed end angles to the right, in FIG. 3C where the closed end angles downward, and in FIG. 3D where the closed end angles upward. It is noted that the directional adjectives of the previous sentence are arbitrary and are defined by the position of the sleeve 302. In certain embodiments, the curvature of the sleeve 302 approximates the curvature of the patient's mouth, whereas in other embodiments, the curvature approximates that found in the mouth of an average patient. The curvature prevents the closed end of the sleeve 302 to bore into, or unduly rub against the inside of the patient's cheeks, and allows for greater comfort for the patient when the device 100 is worn.

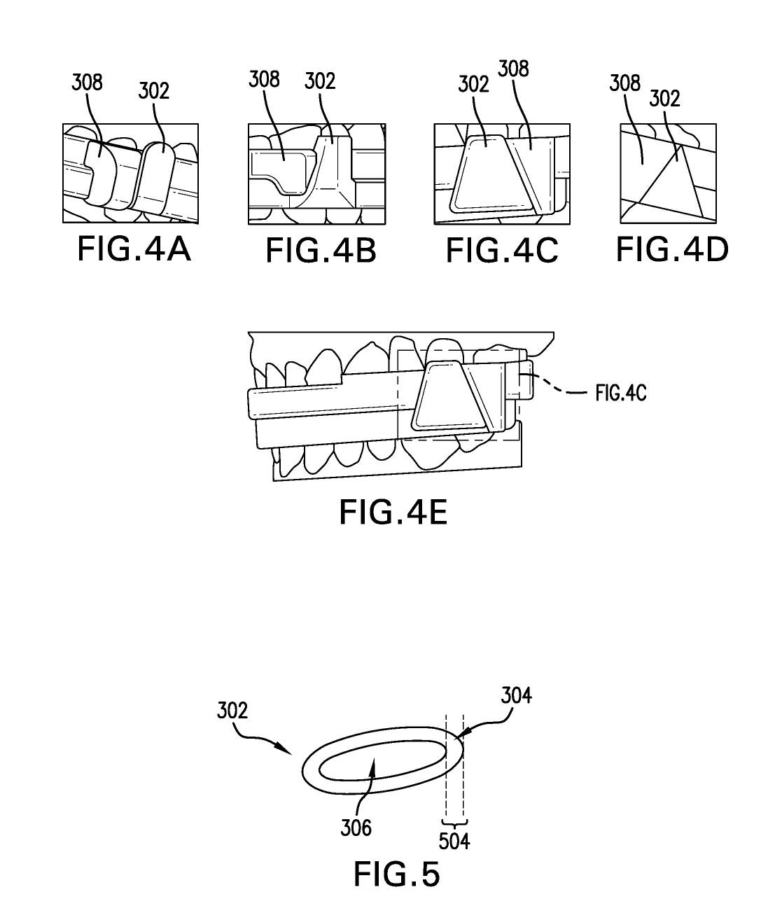

The sleeve 302, and the corresponding sleeve 308 for the upper sleeve, can each have one of a multitude of designs and shapes. In one embodiment, the sleeves are selected from a predesigned digital library of sleeves. FIGS. 4A-4D show a non-exhaustive variety of the design embodiments used in a sleeve library. In some embodiments, a designer calls from a library of sleeve designs, for example those shown in FIGS. 4A-4D, and selects one sleeve type (for example that shown in FIG. 4C). The design is selected based on the patient's need and the geometry of the patient's dentition and mouth. A set of sleeves 302 are then prepared having the desired sleeve design. The sleeve 302 is then placed on the fin 102 of the customized patient splint design, as shown in FIG. 4E.

FIG. 5 shows the bottom view of the sleeve 302. The outer shell 304 of the sleeve 302 has a thickness 504. In some embodiments, the thickness 504 is uniform throughout the perimeter of sleeve 302. In other embodiments, the thickness 504 varies from location to location in order to enhance the strength of the sleeve 302. In certain embodiments, at least the thickness 504 along the contact surface of sleeve 302 with sleeve 308 varies from one sleeve to another in a set of multiple sleeves prepared for the same patient. Thus, by varying the thickness 504 of either or both of sleeves 302 and 308, the extent of mandibular advancement is varied.

In some embodiments, the thickness 504 can be varied, either within a sleeve or from one sleeve to another, for example, from a 1 nm to 5 mm, or from 1 μm to 5 mm, or from 1 mm to 5 mm. In some embodiments, the thickness is no more than 4 mm, 3 mm, 2 mm, or 1 mm.

Various locking mechanisms are contemplated to secure the sleeve 302 over the fin 102. In some embodiments, the sleeve 302 is held in place over the fin 102 by a friction lock mechanism. In these embodiments, the tight fit of the sleeve 302 over the fin 102 creates enough friction that the normal use of the device does not dislodge the sleeve 302 from over the fin 102. In some of these embodiments, corresponding grooves (not shown) on one or both of the sleeve 302 and fin 102 increases the friction between the two pieces.

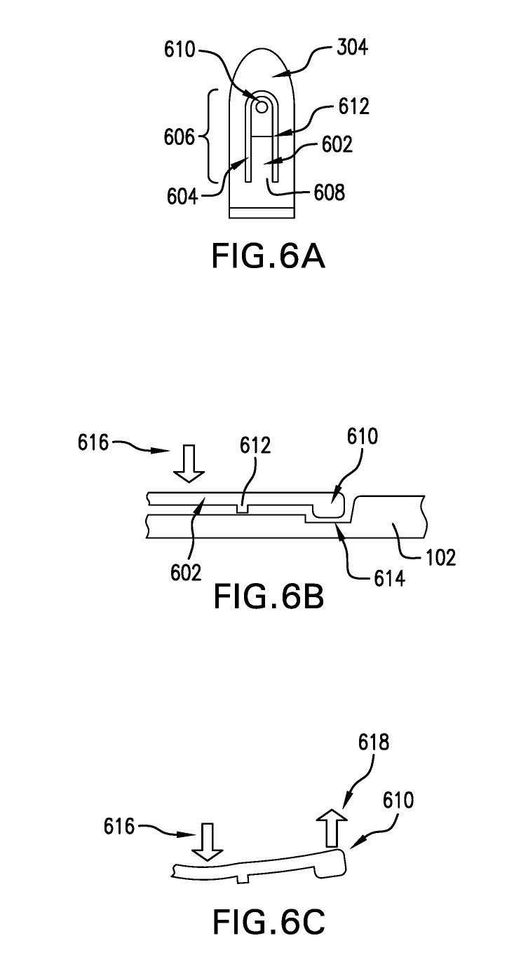

In other embodiments, such as the one shown in FIGS. 6A-6C, the locking mechanism is a key-tab mechanism. As depicted in FIG. 6A, the tab mechanism 602 is incorporated into the design of the sleeve 302. The tab mechanism 602 is separated from the sleeve shell 304 by a gap 604. The thickness of the gap 604 can be varied, for example, from a 1 nm to 5 mm, or from 1 μm to 5 mm, or from 1 mm to 5 mm. The tab 602 has a length 606, which is less than the full length of the sleeve 302. The length 606 can be varied depending on the thickness 504 of the shell 304, or the hardness of the material making up the sleeve 302, and in some instances depending on the dexterity of the patient, to provide for a convenient release operation, as discussed below.

At one end of the tab, either the end close to the open end or the end close to the closed end of the sleeve 302, the tab 602 is connected to the sleeve shell 304 by a living hinge 608. In the embodiment shown in FIG. 6A, the living hinge 608 is proximal to the open end of the sleeve 302. In some embodiments, the tab 602 comprises a key 610 at the opposite end of the tab 602 from the living hinge 608. Along the length 606, and between the living hinge 608 and the key 610, a fulcrum 612 is located. The position of the fulcrum 612 can be varied to provide the most convenient release operation for the patient.

FIG. 6B shows a close up view of the tab 602 when the sleeve 302 is placed over the fin 102. In some embodiments, the fin 102 comprises a notch 614. When the sleeve 302 is placed over the fin 102, the key 610 fits into the notch 614, thereby holding the sleeve 302 in place. To release the sleeve 302, the user pushes on the tab 602 at a location between the fulcrum 612 and the living hinge 608 in the direction of arrow 616. As shown in FIG. 6C, when the tab 602 is pressed in the direction 616, the key 610 moves in the opposite direction 618 and the key 610 is released from the notch 614, allowing the sleeve 302 to be removed.

In some embodiments, the tab 602 is located on the lingual side of the sleeve 302 (i.e., the side facing the mouth cavity, or the tongue), whereas in other embodiments, the tab 602 is located on the buccal side of the sleeve 302 (i.e., the side facing the inside of the patient's cheek). In some embodiments, the sleeve 302 comprises at least two tabs 602, one on the lingual side and one on the buccal side. In other embodiments, the tab (or tabs, if there are more than one tab) 602 are located on the surfaces orthogonal to the lingual and buccal surfaces. In certain embodiments, the tab 602 is located on the surface opposite the contact surface. The presence of more than one tab 602 provides additional locking strength.

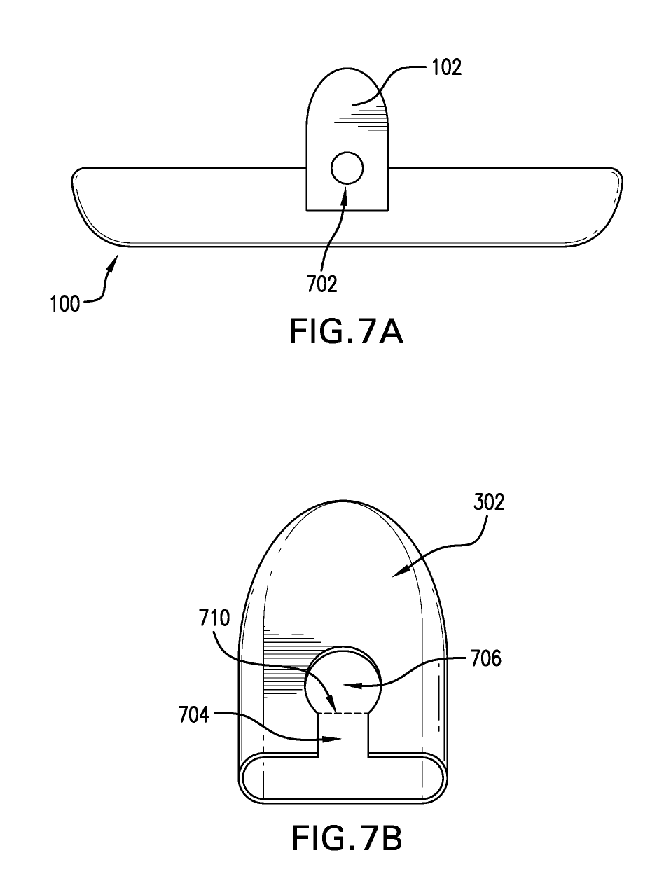

FIGS. 7A-7B show another embodiment of a locking mechanism. The fin 102 on the splint 100 comprises a key button 702, which is a raised boss, as shown in FIG. 7A. The sleeve 302, shown in FIG. 7B, comprises a key way 704, which culminates in a key hole 706. The shape of the key hole 706 matches the approximate contours and size of the key button 702. In the embodiment shown in FIGS. 7A & 7B, the shape of the key button 702 and the key hole 706 is approximately circular. As shown in FIG. 7B, the key way 704 opens at the open end of the sleeve 302. The threshold 710 of the key hole 706 is marked in FIG. 7B by an imaginary dashed line. The threshold 710 provides a friction lock for the key button 702 such that once the sleeve 302 is placed over the fin 102 and the key button 702 is placed inside the key hole 706, the sleeve 302 does not fall out of place without the user intentionally removing the sleeve 302.

As discussed above, in the '208 application, and shown in FIGS. 4A-4D, the rake angle can be modified to be in either neutral, procline, or recline orientation. In some embodiments, the fin 102 is in a neutral orientation and the rake angle is changed by changing the sleeve 302. In these embodiments, only the sleeve 302 affords a change in the rake angle. In other embodiments, both the fin 102 and the sleeve 302 are oriented in the desired rake angle orientation.

In one aspect, provided herein are nesting sleeves. In some embodiments, different sleeves have different sized hollow interior 306, such as one sleeve can fit over another sleeve. To titrate the patient, first the smallest of the selected sleeves (a “first sleeve”) is put over the fin and the device is tested. if there is a desire to increase the extent of mandibular advancement, then another sleeve, with a larger hollow interior 306 (a “second sleeve”), is placed over the first sleeve, thereby increasing the overall thickness covering the fin. The next sleeve in the set (a “third sleeve”) can fit over the second sleeve and increase the thickness yet again.

In some of these embodiments, the thickness 504 of the different sleeves is the same, whereas in other embodiments, the thickness 504 of one sleeve is different than the thickness 504 of another sleeve. In certain embodiments, the thickness 504 of the second and subsequent sleeves on the buccal and lingual sides is kept relatively thin, i.e., 75%, 50%, 40%, 25%, or 10%, of the thickness 504 of the sleeve on the contact surface side. By varying the thickness in this manner, the sleeve bulk in the patient's mouth is kept to a minimum while the mandibular advancement is increased.

In some embodiments, the sleeves having different thickness 504 or different sized hollow interior 306, have different colors. In certain embodiments, the sleeves are opaque whereas in other embodiments, the sleeves are transparent. In some embodiments where the second sleeve fits over the first sleeve, as discussed above, the sleeves are both transparent and have different colors. In certain of these embodiments, the colors of the first and second sleeves combine to form a new color. For example, and without limitation, in one embodiment the first sleeve is blue and the second sleeve is red. When the second sleeve is placed over the first sleeve, then the combined color will be purple. In these embodiments, the patient or the healthcare provider can quickly determine the extent of mandibular advancement by looking at the color of the sleeved fin.

In another aspect, disclosed herein are methods of treating a patient with a mandibular advancement device (MAD), the method comprising:

identifying a patient in need thereof;

providing the patient with a set of one upper splint and one lower splint of the MAD, each splint having at least one fin;

providing the patient with a plurality of sleeves for each fin, comprising a first sleeve and a second sleeve for each fin;

instructing the patient to place the first sleeve of each fin over the corresponding fin and use the device to advance the mandible;

if the result of using the MAD with the first sleeve was unsatisfactory, then instructing the patient to place the second sleeve of at least one of the fins over the first sleeve of the corresponding fin, place the combination over the corresponding fin, and use the device to advance the mandible.

In another aspect, disclosed herein are methods of treating a patient with a mandibular advancement device (MAD), the method comprising:

identifying a patient in need thereof;

providing the patient with a set of one upper splint and one lower splint of the MAD, each splint having at least one fin;

providing the patient with a plurality of sleeves for each fin, comprising a first sleeve and a second sleeve for each fin;

instructing the patient to place the first sleeve of each fin over the corresponding fin and use the device to advance the mandible;

if the result of using the MAD with the first sleeve was unsatisfactory, then instructing the patient to place the second sleeve of at least one of the fins over the corresponding fin and use the device to advance the mandible.

What is claimed is:

1. A sleeve for use with a fin of a mandibular advancement device, the sleeve comprising:
a shell, having a wall defining a hollow interior,
wherein the wall encloses the hollow interior on all sides except one, leaving an opening at one end of the body;
the wall has a thickness in the range of from about 1 nm to about 5 mm;
the hollow interior comprises approximately the same size and dimensions as the fin of the mandibular advancement device;
a locking mechanism;
the sleeve has a rake angle and the fin has a rake angle, wherein the rake angle of the sleeve is the same as, or different than, the rake angle of the fin, and wherein the rake angle of the sleeve is between about 20° to about 80°, or is between about 100° to about 160°;
the closed end of the sleeve is curved away from the plane normal to the plane defined by the rim of the open end.

2. A mandibular advancement device comprising:
a splint;
at least one fin connected to the splint;
a sleeve configured to fit over the fin, the sleeve comprising:
a shell, having a wall defining a hollow interior,
wherein the wall encloses the hollow interior on all sides except one, leaving an opening at one end of the body;
the wall has a thickness in the range of from about 1 nm to about 5 mm;
the hollow interior comprises approximately the same size and dimensions as the fin of the mandibular advancement device.

3. A sleeve for use with a fin of a mandibular advancement device, the sleeve comprising:
a shell, having a wall defining a hollow interior,
wherein the wall encloses the hollow interior on all sides except one, leaving an opening at one end of the body;
the wall has a thickness in the range of from about 1 nm to about 5 mm;
the hollow interior comprises approximately the same size and dimensions as the fin of the mandibular advancement device.

4. The sleeve of claim 3, wherein the sleeve has a rake angle and the fin has a rake angle, and the rake angle of the sleeve is the same as, or different than, the rake angle of the fin.

5. The sleeve of claim 2, wherein the rake angle is between about 20° to about 80°, or is between about 100° to about 160°.

6. The sleeve of any one of claims 3-5, wherein when worn over the fin, the open end of the sleeve abuts the splint, whereas the closed end is distal to the splint.

7. The sleeve of any one of claims 3-6, wherein the fit between the sleeve and the fin is such that when the sleeve is placed over the fin, the sleeve is substantially immobile with respect to the fin.

8. The sleeve of any one of claims 3-7, wherein the closed end of the sleeve is curved away from the plane normal to the plane defined by the rim of the open end.

9. The sleeve of any one of claims 3-8, wherein the thickness of the wall is uniform throughout the perimeter of the sleeve.

10. The sleeve of any one of claims 3-9, wherein the thickness of the wall varies from location to location on the sleeve.

11. The sleeve of any one of claims 3-10, wherein the sleeve comprises a locking mechanism.

12. The sleeve of claim 11, wherein the locking mechanism is a friction lock mechanism.

13. The sleeve of claim 12, wherein the sleeve comprises one or more grooves to increase the friction.

14. The sleeve of claim 11, wherein the locking mechanism is a key-tab mechanism.

15. A plurality of sleeves each according to claim 1, wherein the thickness of the wall varies from one sleeve to another.

16. The plurality of sleeves of claim 15, wherein one sleeve nests within another sleeve, and the combination of nested sleeves fits over the fin of the mandibular advancement device.

17. A method of treating a patient with a mandibular advancement device (MAD), the method comprising:
identifying a patient in need thereof;
providing the patient with a set of one upper splint and one lower splint of the MAD, each splint having at least one fin;
providing the patient with a plurality of sleeves for each fin, comprising a first sleeve and a second sleeve for each fin;
instructing the patient to place the first sleeve of each fin over the corresponding fin and use the device to advance the mandible;
if the result of using the MAD with the first sleeve was unsatisfactory, then instructing the patient to place the second sleeve of at least one of the fins over the first sleeve of the corresponding fin, place the combination over the corresponding fin, and use the device to advance the mandible.

18. A method of treating a patient with a mandibular advancement device (MAD), the method comprising:
identifying a patient in need thereof;
providing the patient with a set of one upper splint and one lower splint of the MAD, each splint having at least one fin;
providing the patient with a plurality of sleeves for each fin, comprising a first sleeve and a second sleeve for each fin;
instructing the patient to place the first sleeve of each fin over the corresponding fin and use the device to advance the mandible;
if the result of using the MAD with the first sleeve was unsatisfactory, then instructing the patient to place the second sleeve of at least one of the fins over the corresponding fin and use the device to advance the mandible.
